(19) United States
(12) Patent Application Publication
(10) Pub. No.: US 2017/0055952 A1
(43) Pub. Date: Mar. 2, 2017

(54) IMAGER TOUCH PANEL WITH WORKSHEET AND CONTROL REGIONS FOR CONCURRENT ASSESSMENT DOCUMENTING AND IMAGER CONTROL

(71) Applicant: Ultrasonix Medical, Richmond (CA)

(72) Inventors: Hani Eskandari, Vancouver (CA); Tomas Bobovsky, North Vancouver (CA); Linda Pendziwol, Nanaimo (CA)

(21) Appl. No.: 14/837,667

(22) Filed: Aug. 27, 2015

(51) Int. Cl. A61B 8/00; G06F 19/00; G06F 3/0488; G06F 3/041; G06F 3/0484

(52) U.S. Cl. CPC A61B 8/465; G06F 3/0416; G06F 3/04847; G06F 3/04886; G06F 19/3406; G06F 19/321; A61B 8/463; A61B 8/467

(57) ABSTRACT

A method includes concurrently activating a worksheet region and a controls region concurrently present on the touch sensitive user interface. The method further includes receiving a signal indicating the at least one control of the controls region is selected through a touch on a touch sensitive area of the controls region corresponding to the control and controlling, in response to the signal, the at least one of the scanning by the ultrasound imager and the ultrasound imager parameter of the ultrasound imager while maintaining the activation of both the concurrently present worksheet and controls regions. The method further includes receiving a first input at an editable field of the digital worksheet form in the worksheet region and entering the first input into the editable field, which transforms and constructs an updated digital worksheet form while maintaining the activation of both the concurrently present worksheet and controls regions.

### TECHNICAL FIELD

The following generally relates to ultrasound imaging and more particular to a touch panel, of an ultrasound imager, with user interactive touch sensitive worksheet region and a controls region for concurrent assessment documenting, and ultrasound imager control.

### BACKGROUND

Some clinical applications for ultrasonography, such as emergency medicine (EMED), require performing a fast diagnostic imaging procedure on a patient and subsequently reporting the findings as quickly as possible. The reporting has been through formal reports and worksheets. Formal reports are produced by the machine that includes measurements and possibly physician's diagnosis, and worksheets generally are informal reports, which are filled in by the technician (sonographer) to be later reviewed by the physician to make the physician aware of the technician's assessment of the scan. An example of a sub-portion of a worksheet from a 2011 ACEP (American College of Emergency Physicians) guideline is shown in FIG. 1.

In applications such as EMED, the speed at which the diagnosis is performed can be critical for treating the patient effectively and managing the queue of patients in the emergency facility. In current practice, the clinician either performs an ultrasound examination and then retrieves and fills in the worksheet (sequential workflow) or pauses an ongoing ultrasound examination and partially fills in the worksheet as needed (interlaced workflow). Furthermore, with a paper worksheet, the clinician may manually hand draw a diagram of the scanned anatomical tissue or organ on the paper and mark a location of tissue of interest such as a suspected lesion on the anatomical drawing.

With the former approach, the clinician, after the examination, moves to a reporting area and has to re-visit images already observed and may not remember an earlier observation. Furthermore, the clinician has to spend added time outside of the actual examination to create the report, which delays diagnosis. With the latter, moving back and forth between the examination and a worksheet increases examination time, which delays diagnosis, and the clinician has to keep track of what has been performed so that a particular image is not missed or acquired more than once.

### SUMMARY

Aspects of the application address the above matters, and others.

In one aspect, a method includes concurrently activating a worksheet region and a controls region of a touch sensitive user interface. The worksheet region visually displays a digital worksheet form with fields editable through touch sensitive areas of the worksheet region. The controls region visually displays at least one control that controls at least one of scanning by an ultrasound imager and an ultrasound imager parameter of the ultrasound imager. The concurrently activated worksheet and controls regions are concurrently present on the touch sensitive user interface. The method further includes receiving a signal indicating the at least one control of the controls region is selected through a touch on a touch sensitive area of the controls region corresponding to the control while maintaining the activation of both the concurrently present worksheet and controls regions. The method further includes controlling, in response to the signal, the at least one of the scanning by the ultrasound imager and the ultrasound imager parameter of the ultrasound imager while maintaining the activation of both the concurrently present worksheet and controls regions. The method further includes receiving a first input at an editable field of the digital worksheet form in the worksheet region while maintaining the activation of both the concurrently present worksheet and controls regions. The method further includes entering the first input into the editable field, which transforms and constructs an updated digital worksheet form while maintaining the activation of both the concurrently present worksheet and controls regions. Those skilled in the art will recognize still other aspects of the present application upon reading and understanding the attached description.

In another aspect, an ultrasound imaging system includes circuitry configured to control ultrasound transducer element excitation and echo reception by an ultrasound transducer of the imaging system, processing of received echoes, and ultrasound image display. A user interface with a touch sensitive region, which includes at least a worksheet region and a controls region, wherein the worksheet region is configured to visually display a digital worksheet form with fields editable through touch sensitive areas of the worksheet region, and the controls region is configured to display at least one touch sensitive soft control, and wherein the worksheet region and the controls region are concurrently present on the touch sensitive region. A user interface controller configured to control the touch sensitive region, including forms displayed in the worksheet region and controls displayed in the controls region and generating signals in response to sensing activation of a touch sensitive area of at least one of a displayed form in the worksheet region and a displayed control in the controls region, and further configured to concurrently activate the worksheet region and the controls region so that the editable digital worksheet form is editable in the worksheet region concurrently while the at least one touch sensitive soft control is selectable in the controls region.

In another aspect, a method includes visibly presenting a touch sensitive worksheet region on a user interface, wherein the worksheet region visually displays a digital worksheet form with fields editable through touch sensitive areas of the worksheet region, receiving an edit input at a location over a sub-portion of a visually displayed graphical representation of anatomy of interest in the worksheet region, and displaying selected graphical indicia at the location in response to receiving the edit input at the location.

### BRIEF DESCRIPTION OF THE DRAWINGS

The application is illustrated by way of example and not limitation in the figures of the accompanying drawings, in which like references indicate similar elements and in which:

FIG. 1 schematically a sub-portion of a worksheet from the 2011 American College of Emergency Physicians (ACEP) guidelines;

FIG. 2 schematically illustrates an example ultrasound imaging system with a touch screen user interface with a worksheet region and a control region;

FIG. 3 illustrates an example of the worksheet region and a control region of the touch screen user interface;

FIG. 4 illustrates another example of the touch screen user interface in which the worksheet region and regions are move and/or resized;

FIG. 5 illustrates another example of the touch screen user interface in which one of the regions overlaps another region;

FIG. 6 illustrates another example of the touch screen user interface in which one of the regions is visibly hidden;

FIG. 7 illustrates another example of the touch screen user interface in which one of the regions is replaced by another region;

FIG. 8 illustrates another example of the touch screen user interface in which one of the regions is minimized;

FIG. 9 illustrates another example of the touch screen user interface in which one or more other regions are concurrently displayed;

FIG. 10 illustrates another example of the touch screen user interface in which the regions are of equal size and occupy the entire display;

FIG. 11 illustrates another example of the touch screen user interface in one of the regions occupies the entire display;

FIG. 12 illustrates an example of a sub-portion of a worksheet with checkboxes;

FIG. 13 illustrates an example of a sub-portion of a worksheet with a graphical anatomical representation; and

FIG. 14 illustrates example method in accordance with the description herein.

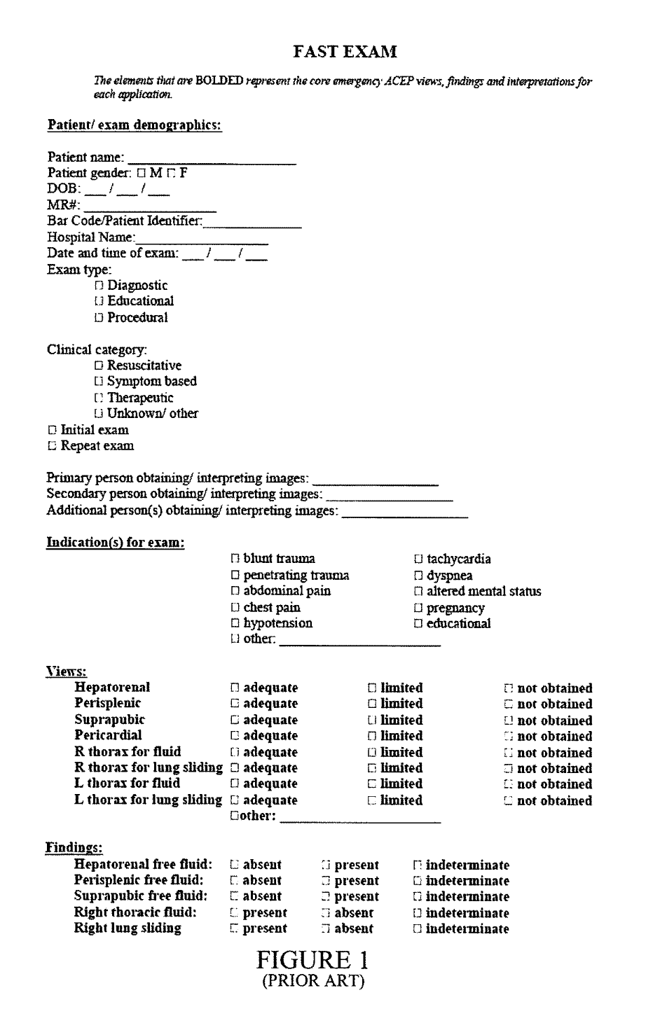

### DETAILED DESCRIPTION

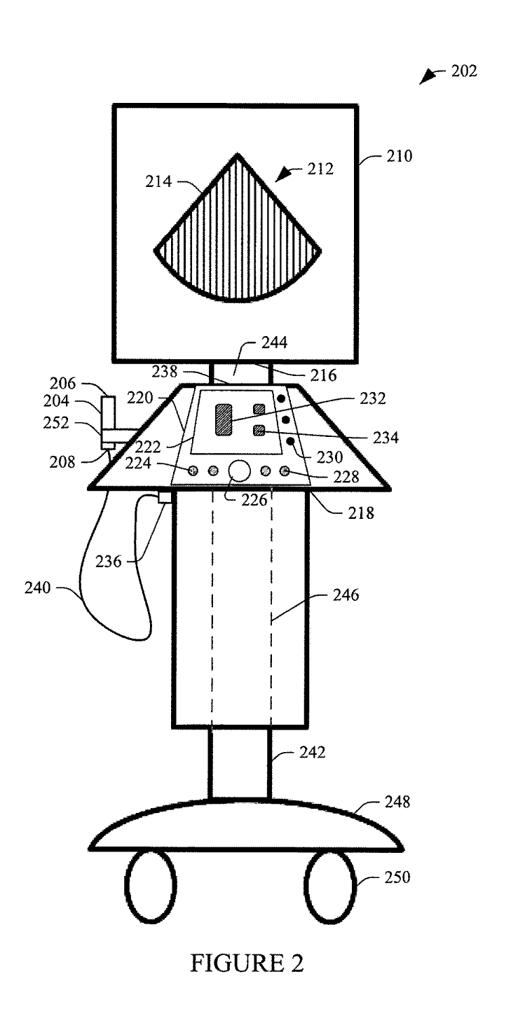

FIG. 2 schematically illustrates an ultrasound (US) imager or imaging system 202.

The ultrasound imaging system 202 includes a probe 204 with a one-dimensional (1D) or two-dimensional (2D) transducer array 206 with at least one transducer element. Suitable array configurations include, but are not limited to, linear, curved (e.g., concave, convex, etc.), circular, etc., full populated or sparse, etc. The probe 204 further includes a console interface 208, which may include a physical connector (e.g., an electro-mechanical device for joining electrical circuits) and/or wireless transceiver.

The ultrasound imaging system 202 further includes a display monitor 210. The display monitor 210 can be a cathode ray tube (CRT), a liquid crystal display (LCD), a light emitting diode (LED), and/or other display monitor. The display monitor 210 includes at least one image display area 212 configured to display at least one image 214. The display monitor 210 further includes a console interface 216, which may include a physical connector (e.g., an electro-mechanical device for joining electrical circuits) and/or wireless receiver.

The ultrasound imaging system 202 further includes a console 218. The console 218 houses components (not visible) such as transmit and receive circuitry that controls excitation of the at least one transducer element and detection of echoes with the at least one transducer element, a beamformer that processes signals indicative of the detected echoes and creates scanlines, a scan converter that converts the beamformed data to data viewable via the monitor 210, a processor (cpu, microprocessor, controller, etc.) and/or other components such as those for Doppler, flow velocity, etc. processing.

The console 218 further includes a user interface 220. The user interface 220 includes at least a touch sensitive region 222 with a touch sensitive surface. In the illustrated example, the user interface 220 further includes physical (“hard”) controls such as one or more knobs 224, dials 226, buttons 228, trackball 230, and/or other physical controls. Other user interface features may include visual indicators (e.g., lights, etc.), audible indicators (e.g., speakers, etc.), tactile indicators (e.g., a rough surface, a recess, etc.), and/or other indicators.

The touch sensitive region controls and/or the physical controls may include a time-gain control (TGC), a mode selection control (e.g., 2D, 3D, 4D, pulse wave (PW) Doppler, Color (Col) Doppler, M, etc.), a CINE control, a measurement control, a parameter control (e.g., zoom, a depth, focus, etc.), etc. For the touch sensitive region 222, a location of one or more of the controls may be moveable within the region, a visibility of the one or more of the controls within the region may be selectively, controls can be grouped together, etc.

As described in greater detail below, the touch sensitive region 222 at least includes a display workspace 232 for, e.g., selecting and/or entering information in a digital worksheet displayed in the workspace 232, and one or more soft or touch controls 234, where the digital worksheet displayed in the workspace 232 and the one or more soft or touch controls are concurrently active such that the digital worksheet can be edited and/or modified in the workspace 232 while an active control controls scanning and/or an ultrasound parameter.

The touch sensitive region 222 may include a resistive, a capacitive, etc. region. Furthermore, the touch sensitive region 222 may include a LCD, thin film transistor (TFT) LCD, organic LED (OLED), and/or other technology. Examples of suitable user interfaces include, but are not limited to, PCT/IB2014/066363, entitled “Ultrasound Imaging System Touch Panel with Multiple Different Clusters of Controls,” and filed Nov. 26, 2014, and PCT/IB2014/066365, entitled “Ultrasound Imaging System Touch Panel Cluster Control Interaction,” and filed Nov. 26, 2014, the entireties of which are incorporated herein by reference.

The console 218 further includes a probe interface 236 and a display monitor interface 238, which respectively are complementary to the console interfaces 208 and 216. For example, the probe interface 236 is complementary to the console interface 208, and, in the illustrated example, are in electrical communication via a cable 240 connected there between. The cable 240 is fixedly attached to the console interface 208 and removeably attached to the probe interface 236, which may be through female and male connectors that physically engage and physically connect electrodes.

In this example, the ultrasound imaging system 202 includes a mobile mechanical support 242 with a monitor support 244, a console support 246, and a base 248 with movers 250 such as one or more wheels, casters, etc. The monitor 210 is attached to the monitor support 244 and the console 218 is attached to the console support 246. A probe support 252 is configured to support the probe 204. In a variation, the console 218 rests on a table, a desk, a stationary cart, etc. with the monitor 210 either part of the console (e.g., similar to a laptop) or a separate device in electrical communication therewith.

FIG. 3 schematically illustrates an example of the touch sensitive region 222.

The touch sensitive region 222 includes a worksheet region 302 (e.g., the display workspace 232 in FIG. 2) and a controls region 304. The worksheet region 302 occupies a first sub-portion of the touch sensitive region area and the controls region 304 occupies a first sub-portion of the touch sensitive region area, and the first and second sub-portions do not occupy the entire the touch sensitive region area. At least one of the regions 302 and/or 304 may be fixed, moveable, re-shapeable, and/or re-sizeable.

As briefly discussed above, the worksheet region 302 is configured to display an editable digital worksheet. Editable means the worksheet region 302 can display a form with pre-determined regions which can be selected to toggle between pre-determined options, e.g., a check box that can be toggled between “checked” and “unchecked” states. Editable may also mean a pre-determined region can be activated to allow alpha-numeric and/or graphic indicia entry via a soft and/or physical keyboard, a gesture on the touch screen (e.g., via one or more fingers, a stylus, a digital pen, etc.), voice recognition, etc. The term editable is not limited to these example definitions.

The controls region 304, as briefly discussed above, includes one or more soft controls. The displayed and/or activated soft controls may be grouped, e.g., soft keys that control the imaging parameters may be grouped together. Such controls may become visible and/or activated depending on the point in examination (e.g., protocol and/or parameter selection, scanning, taking measurements, etc.). The control groupings can be default, facility specific, user specific, scanning mode specific, etc.

With the touch sensitive region 222 of FIG. 3, the sonographer can concurrently scan and/or change parameters using the controls in the controls region 304 and edit a worksheet displayed in the worksheet region 302. This may improve diagnosis accuracy with respect to scanning and completing the worksheet and reporting the results, relative to a configuration in which the either scan and/or change parameters are controlled or the worksheet is edited. For example, the worksheet report may be more reliable as it is generated and completed at the same time as the diagnosis is made.

This also allows for reduction in the aggregate time of scanning and reporting by the clinician, relative to a configuration in which the sonographer would either halt the imaging or end the exam in order to complete or partially fill in the worksheet or report. For example, the overall procedure time may be decreased by as much as several minutes. As such, the system 102 is well-suited for examinations that require quick diagnosis and reporting, such as those for emergency medicine ultrasound. Moreover, this reduces the amount of time a patient has to wait for a scan.

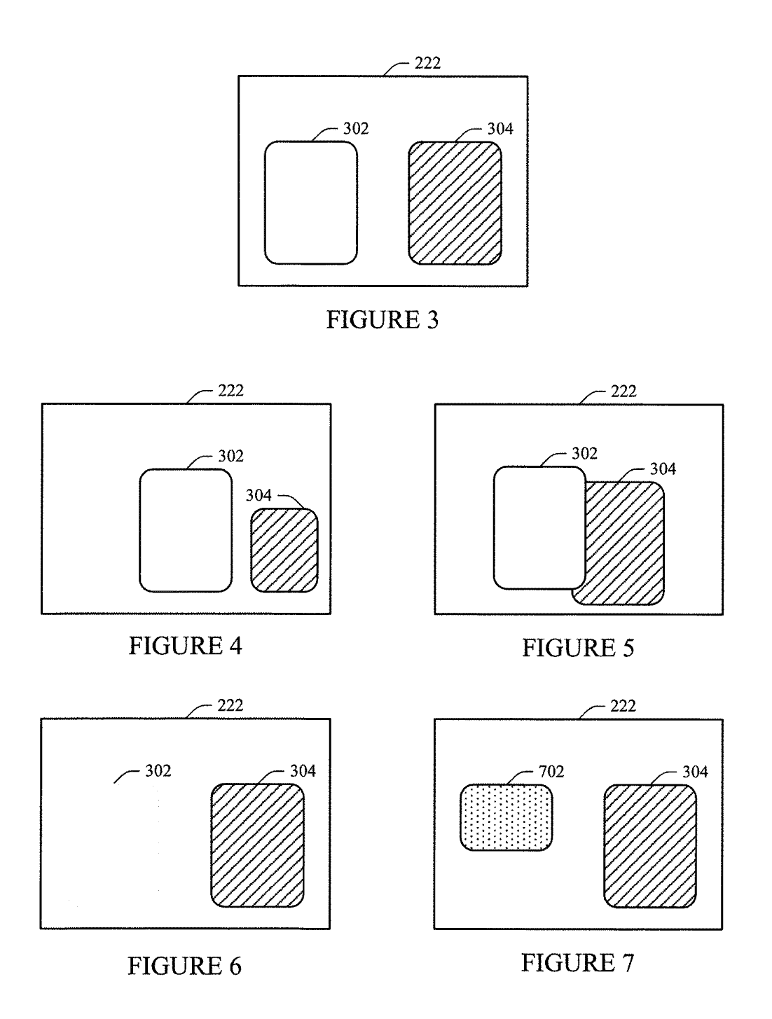

FIG. 4 shows an example in which the controls region 304 is re-sized both of the regions 302 and 304 are moved. FIG. 5 shows an example in which the region 302 partially overlaps the region 304. Re-sizing and/or moving a region can be achieved through soft controls and hard controls (FIG. 2), a pointing device, etc. FIG. 6 shows an example in which the region 302 has been temporality visually hidden. This can also be achieved through the soft controls and/or hard controls (FIG. 2). In FIG. 7, a region 702 is displayed in place of the region 302. The region 702 may display additional (e.g., secondary) controls, an ultrasound image, another worksheet, and/or other information.

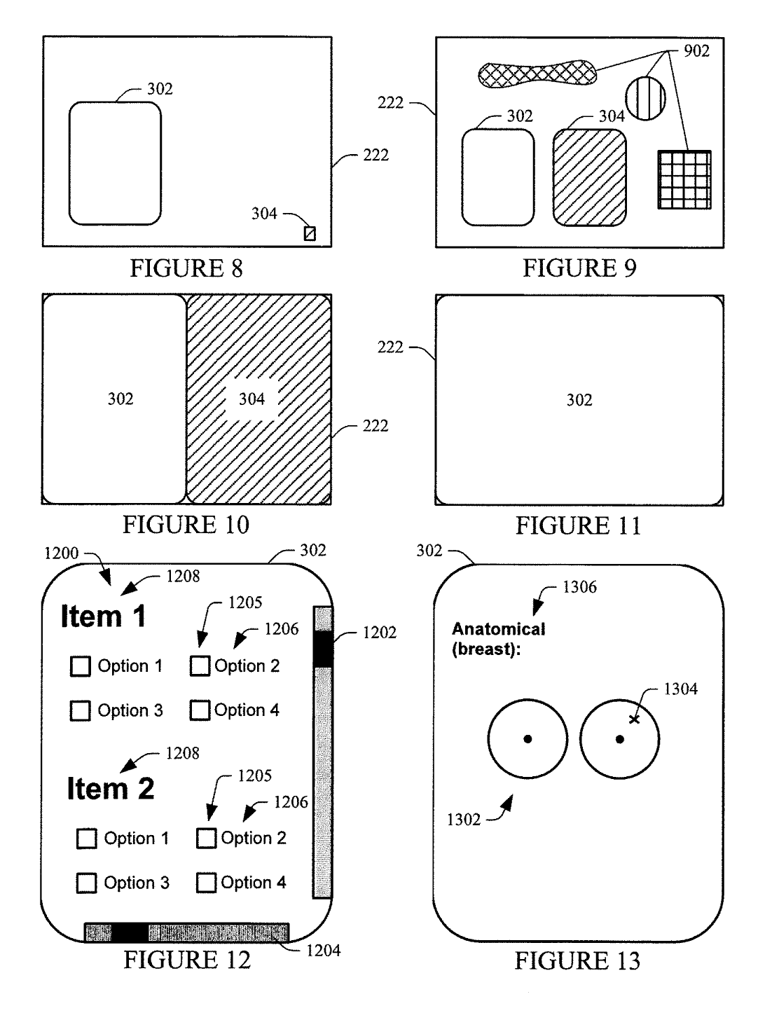

FIG. 8 shows an example in which the region 304 is “minimized” and displayed as a “thumbnail,” a graphical icon, etc., which can be selected (e.g., via a click, a tap on the screen, etc.) to return to the prior display format. FIG. 9 shows an example with at least one more region 902. FIG. 10 shows an example in which the two regions 302 and 304 together occupy the entire area with the two regions 302 and 304 having equal area. FIG. 11 shows an example in which the region occupies the entire area. With this example, the region 304 may be visually hidden behind the region 302, closed, minimized, and/or otherwise displayed or not displayed.

FIG. 12 shows an example in which a sub-portion 1200 of a worksheet is visible in the worksheet region 302. In this example, the worksheet region 302 includes a vertical scroll bar 1202 and a horizontal scroll bar 1204, which allow the sonographer to selectively scroll through the worksheet so that a sub-portion thereof of interest is visible. This may be a current section to fill out, a next section, or a previous already filled out section. This is well suited for instance in which a worksheet and/or report comprises more items than can be reasonably displayed in one region 302.

In one embodiment, the user can scroll the worksheet frame by swiping (e.g., up, down, right, left, diagonal, irregular, etc.) at least one finger, stylus, etc. on the touch screen. Such scrolling action will result in the information that is not initially visible on the screen to become visible. The scrolling may be performed the same way as it may be performed on conventional touch screens such as the ones in most smart phones. In another embodiment, the scrolling action within the worksheet frame can be performed by pressing hard keys, soft keys or soft controls on the touch panel.

In the illustrated example, the visible sub-portion includes checkboxes 1205 next to pre-established options 1206 (e.g., option 1, option 2, option 3, option 4), which are categorized by item 1208 (e.g., item 1, item 2, etc.). In one example, the items 1208 may provide an organ list, an exam list, an image list, etc. and checked options indicate the organ, exam, list, etc. for the examination. Other options may be associated with yes or no conclusions, positive, negative or inconclusive conclusions, etc. Alternative to a list, the options may be in drop down and/or other menus, etc.

It is to be appreciated that the above and/or other changes to the worksheet region 302 can be performed without interrupting or halting the scanning procedure.

FIG. 13 illustrates a sub-portion 1300 of a worksheet visible in the worksheet region 302. This sub-portion 1300 includes a pre-determined representation 1302 (e.g., an anatomical drawing, schematic, etc. of tissue being scanned). The worksheet is editable in that the clinician can place graphical indicia 1304 such as a cross “x”, a plus “+” sign (as shown), a minus “−” sign, an arbitrary sign and/or shapes, etc. on the representation 1302. The clinician can also add annotation 1306 (e.g., text, etc.) on and/or next to the representation 1302.

In the illustrated example, the pre-determined anatomical representation 1302 illustrates left and right breasts, and the graphical indicia 1304 indicates a position of a suspected tumor. In other example, the graphical indicia 1304 may indicate of tissue (e.g., a cyst, scare tissue, previous biopsy site, etc.). In yet another example, the pre-determined anatomical representation 1302 may indicate other tissue, e.g., prostate, liver, kidneys, gallbladder, pancreas, thyroid, heart, muscles, reproductive organs, abdominal organs, etc. A database of anatomical drawings of body organs can be stored in various worksheets or on the ultrasound system 102.

In one embodiment, the clinician can mark the representation 1302 while the scanning is performed (i.e., concurrent use of the regions 302 and 304). The clinician can thus touch within the region 302 to place the graphical indicia 1304 on the representation 1302 and/or move already-placed graphical indicia 1304 to a different location. In another embodiment, the clinician can pause or end the examination and consequently edit the representation 1302 within the worksheet. The edited representation 1302 can be stored as part of the worksheet or report, otherwise stored, discarded, modified, printed, etc.

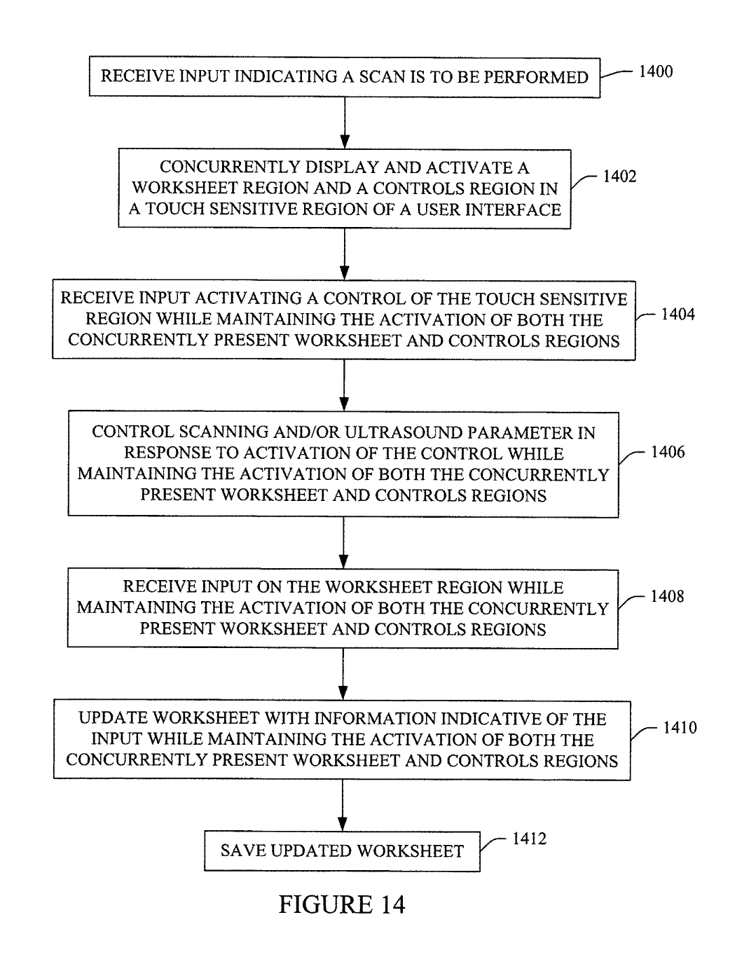

FIG. 14 illustrates example method in accordance with one or more of the embodiments described herein.

It is to be appreciated that the ordering of the below acts is for explanatory purposes and not limiting. As such, other orderings are also contemplated herein. In addition, one or more of the acts may be omitted and/or one or more other acts may be included.

At 1400, an input indicating a scan is to be performed is received.

At 1402, the worksheet region 302 and the controls region 304 are concurrently displayed and activated on the touch sensitive region 222 of the user interface 220.

At 1404, an input by an activated control of the controls region 304 is received while maintaining the activation of both the concurrently present worksheet and controls regions.

At 1406, scanning and/or an ultrasound imaging parameter is controlled in response to the activation of the control while maintaining the activation of both the concurrently present worksheet and controls regions.

At 1408, an input on the worksheet region 304 is received while maintaining the activation of both the concurrently present worksheet and controls regions.

At 1410, a sub-portion of a worksheet displayed via the worksheet region 304 is updated with information indicative of the input while maintaining the activation of both the concurrently present worksheet and controls regions.

At 1412, the updated worksheet is saved. The worksheet and/or the ultrasound data can be stored in a data repository such as a picture archiving and communication system (PACS).

At least a portion of the methods discussed herein may be implemented by way of computer readable instructions, encoded or embedded on computer readable storage medium (which excludes transitory medium), which, when executed by a computer processor(s), causes the processor(s) to carry out the described acts. Additionally or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

What is claimed is:

1. A method, comprising:
concurrently activating a worksheet region and a controls region of a touch sensitive user interface, wherein the worksheet region visually displays a digital worksheet form with fields editable through touch sensitive areas of the worksheet region, wherein the controls region visually displays at least one control that controls at least one of scanning by an ultrasound imager and an ultrasound imager parameter of the ultrasound imager, and wherein the concurrently activated worksheet and controls regions are concurrently present on the touch sensitive user interface;
receiving a signal indicating the at least one control of the controls region is selected through a touch on a touch sensitive area of the controls region corresponding to the control while maintaining the activation of both the concurrently present worksheet and controls regions;
controlling, in response to the signal, the at least one of the scanning by the ultrasound imager and the ultrasound imager parameter of the ultrasound imager while maintaining the activation of both the concurrently present worksheet and controls regions;
receiving a first input at an editable field of the digital worksheet form in the worksheet region while maintaining the activation of both the concurrently present worksheet and controls regions; and
entering the first input into the editable field, which transforms and constructs an updated digital worksheet form while maintaining the activation of both the concurrently present worksheet and controls regions.

2. The method of claim 1, wherein the editable field includes a box and a pre-established option, and wherein the box is configured to toggle between a checked state that indicates selection of the pre-established option and an unchecked state that indicates the pre-established option is not selected in response to receiving touch inputs, and further comprising:
toggling the box to a checked state in response to the edit input while maintaining the activation of both the concurrently present worksheet and controls regions.

3. The method of claim 1, wherein the edit input includes alpha-numeric characters, and further comprising:
entering the alpha-numeric characters into the editable field while maintaining the activation of both the concurrently present worksheet and controls regions.

4. The method of claim 1, further comprising:
receiving the edit input at a location over a sub-portion of a visually displayed graphical representation of anatomy of interest in the worksheet and displaying selected graphical indicia at the location.

5. The method of claim 1, further comprising:
receiving a second input in the worksheet region, wherein the second input indicates an annotation to add to a graphical representation of anatomy of interest in the worksheet region; and
adding the annotation to the graphical representation in the worksheet region while maintaining the activation of both the concurrently present worksheet and controls regions.

6. The method of claim 1, further comprising:
receiving a second input in the worksheet, wherein the second input indicates a change in a location of the digital worksheet form in the touch sensitive user interface; and
moving the worksheet region to the location in the touch sensitive user interface in response to the second input while maintaining the activation of both the concurrently present worksheet and controls regions.

7. The method of claim 6, further comprising: moving the worksheet region to a location that partially overlaps the controls region while maintaining the activation of both the concurrently present worksheet and controls regions.

8. The method of claim 6, further comprising: moving the worksheet region to a location that fully overlaps the controls region while maintaining the activation of both the worksheet and controls regions.

9. The method of claim 1, further comprising:
receiving a second input in the worksheet region, wherein the second input indicates a change in a size of the digital worksheet form in the touch sensitive user interface; and
adjusting the size of the worksheet region in the touch sensitive user interface in response to the second input while maintaining the activation of both the concurrently present worksheet and controls regions.

10. The method of claim 9, further comprising: re-sizing the worksheet region to a full dimension of the touch sensitive user interface while maintaining the activation of both the worksheet and controls regions.

11. The method of claim 1, further comprising:
receiving a second input in the worksheet region; and
visibly hiding the worksheet region in the touch sensitive user interface in response to the second input.

12. The method of claim 11, further comprising:
receiving a third input in the worksheet region; and
visibly unhiding the hidden worksheet region in the touch sensitive user interface in response to the third input while maintaining the activation of both the present worksheet and controls regions.

13. The method of claim 1, further comprising:
displaying a second different controls region in the touch sensitive user interface while maintaining the activation of both the present worksheet and controls regions.

14. The method of claim 1, further comprising:
constructing and outputting a report based on the edited digital worksheet form.

15. An ultrasound imaging system, comprising:
circuitry configured to control ultrasound transducer element excitation and echo reception by an ultrasound transducer of the imaging system, processing of received echoes, and ultrasound image display;
a user interface with a touch sensitive region, which includes at least a worksheet region and a controls region, wherein the worksheet region is configured to visually display a digital worksheet form with fields editable through touch sensitive areas of the worksheet region, and the controls region is configured to display at least one touch sensitive soft control, and wherein the worksheet region and the controls region are concurrently present on the touch sensitive region; and
a user interface controller configured to control the touch sensitive region, including forms displayed in the worksheet region and controls displayed in the controls region and generating signals in response to sensing activation of a touch sensitive area of at least one of a displayed form in the worksheet region and a displayed control in the controls region, and further configured to concurrently activate the worksheet region and the controls region so that the editable digital worksheet form is editable in the worksheet region concurrently while the at least one touch sensitive soft control is selectable in the controls region.

16. The ultrasound imaging system of claim 15, wherein the user interface controller transitions an editable field of the digital worksheet displayed in the worksheet region in an editable mode in response to receiving a touch input at the editable field.

17. The ultrasound imaging system of claim 16, wherein the editable field includes a box and a pre-established option, and wherein the box is configured to toggle between either a checked state that indicates selection of the pre-established option or an unchecked state that indicates the pre-established option is not selected in response to receiving touch input, and the user interface controller toggles the box to the checked state in response to receiving the touch input.

18. The ultrasound imaging system of claim 16, wherein the editable field is configured to receive alpha-numeric characters, and the user interface controller adds received alpha-numeric characters to the editable field in response to receiving the touch input.

19. A method, comprising:
visibly presenting a touch sensitive worksheet region on a user interface, wherein the worksheet region visually displays a digital worksheet form with fields editable through touch sensitive areas of the worksheet region;
receiving an edit input at a location over a sub-portion of a visually displayed graphical representation of anatomy of interest in the worksheet region; and
displaying selected graphical indicia at the location in response to receiving the edit input at the location.

20. The method of claim 19, further comprising:
visibly presenting, concurrently with the touch sensitive worksheet region, a touch sensitive controls region on the user interface, wherein the concurrently presented touch sensitive worksheet and touch sensitive controls regions are concurrently active.
